(19) United States
(12) Patent Application Publication
(10) Pub. No.: US 2024/0216613 A1
(43) Pub. Date: Jul. 4, 2024

(54) SYRINGE BARREL WITH VARYING SILICONE LAYER THICKNESSES, SYRINGE COMPRISING SAME AND USES THEREOF

(71) Applicant: Merz Pharma GmbH& Co. KGaA, Frankfurt (DE)

(72) Inventor: Markus VOGT, Frankfurt (DE)

(21) Appl. No.: 18/292,312

(22) PCT Filed: Jul. 28, 2022
(86) PCT No.: PCT/EP2022/071247

§ 371 Date: Jan. 25, 2024

(30) Foreign Application Priority Data
Jul. 29, 2021 (EP) EP21188553.8

(51) Int. Cl. A61M 5/31; A61M 5/315

(52) U.S. Cl. CPC A61M 5/3134; A61M 5/3135; A61M 5/31505; A61M 2005/3131

(57) ABSTRACT

The present invention relates to a syringe barrel having an interior surface coated with a silicone film having a thickness in the range of 5 nm to 100 nm. It further relates to a method for manufacturing said syringe barrel comprising the step of reducing an initial thickness of a pre-formed silicone film on the interior surface of a syringe barrel to a thickness within the range of about 5 nm to about 100 nm by exposing the syringe barrel to an organic solvent or by subjecting the syringe barrel to mechanical treatment. Furthermore, the present invention relates to a syringe comprising the syringe barrel of the invention, a kit comprising said syringe, and cosmetic and therapeutic uses of said syringe.

### FIELD OF THE INVENTION

The present invention relates a syringe barrel having an interior surface coated with a silicone film having a thickness in the range of 30 nm to 100 nm. It further relates to a method for manufacturing said syringe barrel comprising the step of reducing an initial thickness of a pre-formed silicone film on the interior surface of a syringe barrel to a thickness within the range of about 30 nm to about 100 nm by exposing the syringe barrel to an organic solvent or by subjecting the syringe barrel to mechanical treatment. Furthermore, the present invention relates to a syringe comprising the syringe barrel of the invention, a kit comprising said syringe, and cosmetic and therapeutic uses of said syringe.

### BACKGROUND OF THE INVENTION

Pre-filled syringes have many benefits compared to a vial and a separately provided syringe, such as improved convenience, affordability, accuracy, sterility, and safety. The use of pre-filled syringes results in greater dose precision, in a reduction of the potential for needle sticks injuries that can occur while drawing medication from vials, in pre-measured dosage reducing dosing errors due to the need to reconstitute and/or draw medication into a syringe, and in less overfilling of the syringe helping to reduce costs by minimizing drug waste.

However, a concern for manufacturers of pre-filled syringes is that the syringe functionality is maintained over the entire storage period. More specifically, even after long-term storage of the pre-filled syringe, the gliding force and break loose force of a pre-filled syringe should be neither too low nor too high to ensure the desired convenience, precision and accuracy of injection.

Commercial pre-filled syringes commonly carry an inner wall siliconization layer. This layer facilitates the travel of the plunger stopper through the syringe barrel during use. However, for siliconization there are typically only two different methods used, i.e. spray-on siliconization (also referred to as spray-siliconization) and baked-on siliconization. In the spray-on siliconization method, a silicone oil or a silicone oil emulsion is sprayed into the barrel utilizing a suitable nozzle. Baked-on siliconized barrels then undergo a drying step for thermal fixation of the silicone layer, which makes this layer more resistant against the gliding forces exerted by the moving plunger stopper over time. Both methods lead to discrete levels of siliconization, with the traditional spray-on siliconization leading to much thicker silicone layers.

Syringe manufacturers choosing among the mentioned siliconization methods cannot choose a specific target layer thickness. Although the thickness may vary somewhat from manufacturer to manufacturer, it remains within a certain range depending on the manufacturing process. Spray-on siliconized syringes typically come with a higher layer thickness (e.g., about 300 nm) than baked-on siliconized syringes having a lower layer thickness (e.g., less than 30 nm).

Another concern for manufacturers of pre-filled syringes is the interaction of the filled drug content and the siliconization layer. In fact, the performance of the pre-filled syringe system, i.e., how smooth the plunger stopper can travel along the inner barrel surface, depends on the drug content inside the barrel in combination with the layer thickness. This effect on the performance is observed for both viscous formulations and non-viscous formulations, e.g., aqueous solutions. In many cases the interaction of the formulation and the siliconization layer is a crucial factor for exact dosing. This holds particularly true when, for example, a pre-filled syringe is used for dispensing multiple doses, such as is often the case for the administration of neurotoxins like botulinum toxin and the administration of dermal fillers.

Syringes with enough (thick) inner wall siliconization, e.g., spray-on siliconized syringes, usually allow the plunger to travel smoothly through the barrel even after storage at elevated temperatures and for long storage periods. Baked-on syringes with lower silicone layer thicknesses, however, tend to exhibit increased gliding forces with higher temperature and longer storage time. Thus, if the layer thickness is too low, smooth gliding may no longer be given, resulting in a poor performance of the pre-fille syringe system. The higher the thickness, the better the resulting gliding forces.

However, increased silicone layer thickness is unfortunately accompanied by a higher likelihood of syringe content, in particular protein drug, destabilization since very small silicone droplets migrate from the silicone layer into the formulation over time and adsorb to, or otherwise interact with, ingredients such as protein pharmaceuticals to exert a destabilizing effect.

### OBJECTIVE OF THE INVENTION

In view of the above, the objective of the present invention is to provide a pre-filled syringe which not only has excellent injection force characteristics but also enables stable storage of aqueous formulations such as aqueous protein formulations.

### SUMMARY OF THE INVENTION

The above objective is solved by the provision of a syringe barrel with a silicone film or layer on its inner surface having a targeted siliconization layer thickness that not only enables the provision of a syringe having good syringe functionality in terms of gliding force but also provides a good stability of the syringe content, e.g., of an aqueous formulation, such as an aqueous protein formulation. These favorable mechanical and chemical properties are also achieved upon long-term storage and, thus, makes the syringe barrel well suited for use in the manufacturing of pre-filled syringes, especially pre-filled syringes of sensitive protein formulation such as aqueous botulinum toxin formulations.

In a first aspect, the present invention provides a syringe barrel having a proximal end, a distal end and a sidewall extending therebetween, the sidewall having a cylindrical interior surface defining at least a portion of a chamber for receiving an aqueous formulation (e.g., an aqueous protein formulation), wherein the cylindrical interior surface, e.g. at least a portion of the cylindrical interior surface or the entire cylindrical interior surface, is coated with a silicone layer having a thickness of about 5 nm to about 100 nm.

In a second aspect, the present invention provides a method for manufacturing a syringe barrel of the present invention, comprising steps (a) to (c):
(a) providing a syringe barrel having a proximal end, a distal end and a sidewall extending therebetween, the sidewall having a cylindrical interior surface defining at least a portion of a chamber for receiving an aqueous formulation (e.g., an aqueous protein formulation), wherein the cylindrical interior surface, e.g. at least a portion of the cylindrical interior surface or the entire cylindrical interior surface, is coated with a silicone layer having an initial thickness,
(b) reducing the initial thickness of the silicone layer to between about 30 nm and about 100 nm, particularly to between about 40 nm and about 90 nm, by exposing the cylindrical interior surface of the syringe barrel to an organic solvent (e.g., dichloromethane) or by subjecting the cylindrical interior surface of the syringe barrel to mechanical treatment (e.g., mechanical contact treatment resulting in friction and wear), and
(c) recovering the syringe barrel.

For example, the initial thickness of the silicone layer may be in the range of about 150 nm to about 400 nm, particularly in the range of about 200 nm to 350 nm, and the thickness of the silicone layer after performing method step (c) may be in the range of about 30 nm to about 100 nm, preferably in the range of about 40 nm to about 90 nm.

In a third aspect, the present invention provides a syringe barrel prepared by the method of the present invention.

In a fourth aspect, the present invention provides a syringe comprising the syringe barrel of the present invention. Preferably, the syringe is in the form of a pre-filled syringe containing an aqueous formulation, in particular an aqueous protein formulation such as an aqueous botulinum toxin formulation.

In a fifth aspect, the present invention provides a kit comprising a pre-filled syringe that comprises a syringe barrel of the present invention, and optionally instructions for use of said pre-filled syringe. The pre-filled syringe is preferably filled with an aqueous formulation, in particular an aqueous protein formulation such as an aqueous botulinum toxin formulation.

In a sixth aspect the present invention relates to the use of the syringe of the present invention in therapeutic methods and cosmetic applications.

### DETAILED DESCRIPTION OF THE INVENTION

The present invention provides a syringe barrel with a silicone film on its inner surface having a targeted siliconization layer thickness that not only enables the provision of a syringe having good syringe functionality in terms of gliding force but also provides a good stability of the syringe content, e.g., of an aqueous formulation such as an aqueous protein formulation. These favorable mechanical and chemical properties are also achieved upon long-term storage and, thus, makes the syringe well suited for use as a pre-filled syringe, especially pre-filled syringes of sensitive protein formulation such as botulinum toxin pre-filled syringes.

More specifically, the siliconization layer or film on the inner surface of the syringe barrel is thick enough to result in a low gliding force to allow the stopper to smoothly travel through the syringe barrel during use without the operator having to exert an excessive amount of force. The gliding force is, however, also not too low to result in an undesirable dripping of the syringe content before or after injection or to result in over- or underdosing. As a result, the beneficial mechanical properties associated with the present invention ensures the desired convenience, precision and accuracy of injection. Also, the break loose force (i.e., the force required to initiate the movement of the plunger stopper) is favorably low, especially after long-term storage of the pre-filled syringe, thereby allowing the plunger to move freely when required to do so and without becoming stuck.

On the other hand, the siliconization layer or film on the inner surface of the syringe barrel is thin enough to lower the likelihood of destabilizing of the syringe content, e.g., an aqueous formulation such as an aqueous protein formulation, caused by migration of silicone oil droplets from the siliconization film or layer into the syringe content. Thus, the present invention provides an elevated stability of the syringe content, while maintaining good gliding performance.

In a first aspect, the present invention relates to a syringe barrel having a proximal end, a distal end and a sidewall extending therebetween, the sidewall having a cylindrical interior surface defining at least a portion of a chamber for receiving an aqueous formulation such as an aqueous protein formulation, wherein the cylindrical interior surface, e.g. at least a portion of the cylindrical interior surface or the entire cylindrical interior surface, is coated with a silicone layer having a thickness of about 5 nm to about 100 nm.

Preferably, the silicone layer has a thickness of about 40 nm to about 100 nm, about 40 nm to about 90 nm, about 40 nm to about 80 nm, about 40 nm to about 75 nm, about 40 nm to about 70 nm, about 45 nm to about 100 nm, about 45 nm to about 90 nm, about 45 nm to about 80 nm, about 45 nm to about 75 nm, about 45 nm to about 70 nm, about 50 nm to about 100 nm, about 50 nm to about 90 nm, about 50 nm to about 80 nm, about 50 nm to about 75 nm, about 50 nm to about 70 nm, about 55 nm to about 100 nm, about 55 nm to about 90 nm, about 55 nm to about 80 nm, and about 55 nm to about 75 nm. The silicone layer may also have a thickness of about 5 nm to about 70 nm, about 5 nm to about 50 nm, about 5 nm to about 30 nm, about 10 nm to about 35 nm, or about 15 nm to about 30 nm.

The term “thickness of the silicone layer”, as used herein, means that the silicone layer, at each position of the silicone layer on the cylindrical interior surface, has a thickness within the given range. Preferably, the term “thickness of the silicone layer”, as used herein, means that the silicone layer, at a position of 5 mm, 10 mm, 12 mm or 15 mm from the distal end of the syringe barrel (i.e. the flange) in axial direction but not more than up to 5 mm, 10 mm or 15 mm before the proximal end of the syringe barrel has a thickness lying in the given range. More preferably, the term “thickness of the silicone layer”, as used herein, means that the silicone layer, at a position of 10 mm or more from the flange in axial direction up to 10 mm before the proximal end of the syringe barrel, has a thickness within the given range.

Alternatively, the thickness of the silicone layer may also be defined herein as the average thickness of the silicone layer. The average thickness refers to the mean value of all thicknesses, measured in circumferential direction of the syringe barrel and along the entire length of the barrel in axial direction (i.e. over the entire silicone layer on the cylindrical interior surface of the syringe barrel). In other words, the average thickness is defined as the sum of the thicknesses measured for each measurement point divided by the number of measurement points. The measurement apparatus used in the Examples (Bouncer), for example, makes 270 measurements in total. Within the present invention, the number of measurements is usually 100 or more, preferably 200 or more or 300 or more (e.g., 270). Preferably, the term “average thickness of the silicone layer”, as used herein, means that the silicone layer, at a position of 5 mm, 10 mm, 12 mm or 15 mm from the distal end of the syringe barrel (i.e., the flange) in axial direction up to 5 mm, 10 mm or 15 mm before the proximal end of the syringe barrel has an average thickness lying in the given range. More preferably, the term “average thickness of the silicone layer”, as used herein, means that the silicone layer, at a position of 10 mm or more from the flange in axial direction up to 10 mm before the proximal end of the syringe barrel, has an average thickness within the given range.

In accordance with the present invention, the silicone layer is preferably made by spray-on siliconization and then reducing the thickness of the silicone layer. However, it is also possible and contemplated by the present invention that the silicone layer is made by baked-on siliconization and then reducing the thickness of the silicone layer.

The design of the syringe barrel is not particularly limited and typically has an inside diameter adjusted to accommodate the desired fill volume. More specifically, the dimensions of the syringe barrel are generally such that the ratio between the length of the cylindrical interior surface (L), as measured in axial direction, to the inner diameter (D) of the barrel meets the following relationship: 5≤ L/D≤ 14, preferably 5.5≤ L/D≤ 9.

The syringe barrel is further characterized by having a lumen that has a volume of π×(ID/2)²×L, with ID being the inner diameter of the cylindrical syringe barrel and L being the length of the cylindrical syringe barrel. The volume of this lumen is typically within the range of 0.4 cm³ to 3.8 cm³, preferably between 1.0 cm³ and 3.6 cm³ or between 1.5 cm³ and 3.4 cm³. The actual volume inside the barrel that is available for receiving the aqueous formulation is, however, smaller since the stopper must also fit inside the barrel and, thus, the barrel cannot be filled to its capacity. Commonly used fill volumes are 0.5 ml or 1.0 ml up to 2.5 ml.

The syringe barrel of the present invention may be made of glass or a suitable plastic material such as COP. Within the present invention, the syringe barrel is preferably made of glass.

In accordance with the present invention, the distal end of the syringe barrel generally includes a tip having a passage extending therethrough. The proximal end of the syringe barrel generally includes an outwardly extending flange, i.e. the syringe barrel may include a flange-style interface. The design of the flange may, for example, be compatible with ISO11040. The flange-style interface may further be compatible with an optionally present handle. Furthermore, in case of a Luer-Lock syringe, the syringe may be equipped with a Luer-Lock adaptor of, e.g., polycarbonate.

The term “aqueous formulation”, as used herein, is not particularly limited and may refer to an aqueous solution, aqueous suspension, aqueous dispersion, or aqueous emulsion. In particular, the aqueous formulation may be an aqueous solution. Preferably, the aqueous formulation is an aqueous protein formulation, more preferably an aqueous botulinum toxin formulation, or a soft tissue filler composition. The terms “aqueous protein formulation” and “aqueous botulinum toxin formulation”, as used herein, preferably have the meaning as defined herein further below. The term “soft tissue filler composition”, as used herein, preferably refers to a dermal filler composition that is typically in the form of a hydrogel, particularly a hyaluronic acid (HA)-based dermal filler, more particularly a dermal filler based on crosslinked HA (e.g., BDDE-crosslinked HA), or a calcium hydroxylapatite containing filler, particularly a dermal filler comprising calcium hydroxylapatite suspended in a gel carrier of sterile water, glycerin and sodium carboxymethylcellulose such as Radiesse®, or a filler containing a mixture of HA gel (e.g., BDDE-crosslinked HA gel) and calcium hydroxylapatite particle, or a polysaccharide-protein crosslinked filler, particularly where a cross-linked material is a polysaccharide/fibroin gel, or where the cross-linked material is a hyaluronic acid/fibroin gel (HA/fibroin gel).

The term “about” in the context of numerical values will be readily understood by a person skilled in the art, and preferably means that specific values may be modified by +/−10%. As regards endpoints of ranges, the modifier “about” preferably means that the lower endpoint may be reduced by 10% and the upper endpoint increased by 10%. It is also contemplated that each numerical value or range disclosed in this application can be absolute, i.e., that the modifier “about” can be deleted.

In a second aspect, the present invention relates to a method for manufacturing a syringe barrel of the present invention, the method comprising:
(a) providing a syringe barrel having a proximal end, a distal end and a sidewall extending therebetween, the sidewall having a cylindrical interior surface defining at least a portion of a chamber for receiving an aqueous formulation such as an aqueous protein formulation, wherein the cylindrical interior surface, e.g., at least a portion of the cylindrical interior surface or the entire cylindrical interior surface, is coated with a silicone layer having an initial thickness,
(b) reducing the initial thickness of the silicone layer to between about 5 nm and about 100 nm or between about 30 nm and about 100 nm by exposing the cylindrical interior surface of the syringe barrel to an organic solvent or by subjecting the cylindrical interior surface of the syringe barrel to mechanical treatment, and
(c) recovering the syringe barrel.

In step (a), a siliconized syringe barrel is provided. The syringe barrel may be siliconized, e.g., spray-on siliconized or baked-on siliconized. Spray on siliconization refers to a technique where silicone oil is applied by spraying to the inner surface of the barrel. In the baked-on siliconization method, the applied silicone oil (typically in the form of an emulsion) is subjected to thermal bake. Within the present invention, the provided syringe barrel preferably has a spray-on silicone layer.

The initial thickness of the silicone layer may be in the range of about 110 nm or more, particularly in the range of about 110 to about 400 nm or about 110 nm to about 350 nm, or about 150 nm to about 400 nm or about 200 nm to about 400 nm or about 250 nm to about 400 nm or about 200 nm to about 350 nm or about 150 nm to about 300 nm or about 250 nm to about 400 nm or about 250 nm to about 350 nm.

Preferably, the initial thickness (Di) is Di=target thickness+x, with x being at least about 10 nm to about 395 nm or at least about 10 nm to about 360 nm (e.g., about 20 nm to about 300 nm, or about 30 nm to about 250 nm, or about 50 nm to about 300 nm, or about 100 nm to about 360 nm, or about 200 nm to about 350 nm or about 150 nm to about 350 nm or about 200 nm to about 300 nm) and the target thickness being as defined above, e.g. from about 5 nm to about 100 nm (e.g., about 5 nm to about 70 nm, about 5 nm to about 50 nm, about 5 nm to about 30 nm, about 10 nm to about 35 nm, or about 15 nm to about 30 nm) or from about 30 nm to about 100 nm, particularly from about 40 nm to about 90 nm or from about 45 nm to about 85 nm or from about 50 to about 80 nm.

In step (b), an organic solvent may be used to reduce the initial thickness of the silicone layer. The organic solvent may, for example, be a hydrocarbon solvent, a halogenated hydrocarbon solvent, an ether solvent, a polar aprotic solvent or a polar protic solvent. Preferably, the organic solvent is a hydrocarbon solvent, a halogenated hydrocarbon solvent, an ether solvent or a polar aprotic solvent, more preferably a hydrocarbon solvent or a halogenated hydrocarbon solvent, and most preferably a halogenated hydrocarbon solvent. The halogenated hydrocarbon solvent is preferably a chlorinated hydrocarbon solvent. Particularly preferred, the organic solvent is dichloromethane (DCM or methylene chloride). Particularly suitable organic solvents among the organic solvents mentioned above or below are those having a boiling point in the range of 30° C. to 120° C., preferably in the range of 35° C. to 100° C. or 35° ° C. to 80° C.

Suitable hydrocarbon solvents for use herein include, but are not limited to, hexane, in particular n-hexane, heptane, in particular n-heptane, pentane, in particular n-pentane, benzene, toluene, xylene, solvent naphtha, and cyclohexane.

Halogenated hydrocarbon solvents that may be used within the present invention include, for example, halogenated C₁-C₆ alkyl or halogenated C₂-C₆ alkylene, preferably halogenated C₁-C₄ alkyl or halogenated C₂-C₄ alkylene, more preferably halogenated C₁-C₄ alkyl or halogenated C₁-C₃ alkyl or halogenated C₂ alkyl, wherein “halogenated” preferably means “chlorinated”. Most preferably, the halogenated hydrocarbon is dichloromethane (DCM or methylene chloride) or trichloromethane (TCM or chloroform), particularly DCM.

Suitable ether solvents for use herein include diethyl ether, diisopropylether, hexyl ether, ethyl acetate and butyl acetate.

Examples of usable polar aprotic solvents include ethyl acetate, acetone, methyl ethyl ketone and methyl isobutyl ketone, with ethyl acetate, methyl ethyl ketone and methyl isobutyl ketone being preferred polar aprotic solvents. Exemplary polar protic solvents that may be used herein include formic acid, n-butanol, iso-butanol, sec-butanol, tert-butanol, n-propanol, iso-propanol, ethanol, acetic acid, and 2-ethylhexanol.

In accordance with the present invention, the cylindrical interior surface of the syringe barrel is exposed to the organic solvent for a predetermined time that is sufficient for a given organic solvent to exert its desired effect, i.e., to reduce the thickness of the silicone layer. The contact time may be from seconds to minutes depending on the organic solvent used. For example, the contact time of dichloromethane (DCM) may be in the range of 5 seconds to 300 seconds, particularly in the range of 10 seconds to 120 seconds or 20 seconds to 60 seconds.

In an alternative of step (b), the initial thickness of the silicone layer is reduced by subjecting the cylindrical interior surface of the syringe barrel to mechanical treatment.

The mechanical treatment is carried out using a thickness-reducing component configured to be slidably positioned inside the syringe barrel and providing a friction contact with the silicone layer coated on at least a portion of the cylindrical interior surface, optionally the entire cylindrical interior surface, and wherein the thickness-reducing component is used for performing multiple removal cycles, the multiple removal cycles comprising moving the thickness-reducing component to the distal end of the syringe barrel and then moving the thickness-reducing component to the proximal end of the syringe barrel and optionally removing the thickness-reducing component from the syringe barrel, repeating the moving steps and optional removing step until a silicone layer of the desired thickness within the range of about 5 nm to about 100 nm or from about 30 nm to 100 nm has been formed.

In a preferred embodiment, the thickness-reducing component is a plunger stopper. The plunger stopper is not particularly limited as long as it has a sufficient contact resistance with the siliconized inner surface of the barrel. In order to effect an even reduction of thickness, the size of the plunger is adapted to fit into the barrel such that the contact resistance is preferably the same all around the siliconized inner surface of the barrel. Various different stopper geometries (e.g., stoppers with varying numbers of annular ribs that each contact the inner surface of the barrel) and different materials and/or coatings (e.g., siliconized/fluoropolymer-coated stoppers) can therefore be used within the present invention. The different stoppers may, and likely will, result in different thickness reduction of the silicone layer and, thus, different number of cycles may, and likely will, be required to achieve the same thickness reduction with different stoppers.

Generally, the number of removal cycles to be performed to achieve a desired result in terms of reduced silicone layer thickness may be in the range of 10 to 1000 or 20 to 500 or 30 to 400 or, preferably, may be between 40 and 200 or 50 and 100.

In step (c), the syringe barrel is recovered. The term “recover”, as used herein, does not imply any particular restriction and includes any subsequent steps. For example, in case of mechanical treatment, the empty syringe is removed from a fixing device used for mechanical treatment and can then be used directly in subsequent processing steps. In the case of chemical treatment, the recovering step (c) includes, for example, removing the organic solvent from the barrel, e.g., by emptying the barrel and allowing residual organic solvent to evaporate. If necessary or desired, the residual solvent content may then be determined. In subsequent processing steps, the barrel may be sterilized and/or packed, or a syringe may be assembled from the barrel that is used or packed in the form of an empty syringe. Alternatively, the syringe may be filled with an aqueous formulation (e.g., an aqueous protein formulation), sterilized under moist heat (e.g., autoclaved) and then packed. Another alternative involves aseptic filling into the sterilized barrel/syringe.

The term “comprise”, as used herein, is intended to refer to the open-ended term “include”. Thus, a process that “comprises” steps (a) and (b) does not exclude further steps. However, it is contemplated herein that “comprise” includes the closed term “consist (of)” and, thus, that the term “comprise” may be replaced by “consisting of”, if desired.

In a third aspect, the present invention relates to a syringe barrel prepared by the method of the present invention.

The silicone layer of the syringe barrel has a thickness that is not too high to increase the risk of silicone oil migration into the formulation stored inside the barrel when part of a syringe, i.e., in case of a prefilled syringe filled with an aqueous formulation, especially an aqueous protein formulation such as an aqueous botulinum toxin formulation. At the same time, the silicone layer is thick enough to ensure gliding forces and break-loose forces that are sufficiently low for the use in a syringe, in particular a pre-filled syringe, which allows the convenient administration of aqueous formulations, e.g., an aqueous protein formulation such as an aqueous botulinum toxin formulation.

As used herein, the term “gliding force” generally refers to the force required to sustain the movement of the plunger to expel the content of the syringe, and preferably is defined as the gliding force corresponding to the highest force between 1 mm and 35 mm, more preferably between 2 mm and 35 mm or between 3 mm and 35 mm, plunger displacement distance (distance from flange) in Newton (N). Further, the term “break loose force” is intended to mean the force required to initiate the movement of the plunger or plunger stopper.

In a fourth aspect, the present invention relates to a syringe comprising the syringe barrel of the present invention.

As mentioned above, due to the siliconized barrel of the present invention, the syringe is characterized by a low gliding force, i.e., a low force required to sustain the movement of the plunger to expel the content of the syringe, and a low break loose force, i.e., a low force required to initiate the movement of the plunger.

The configuration of the syringe, which is preferably a pre-filled syringe, is not particularly limited and may further comprise a plunger stopper slidably positioned inside the syringe barrel and providing a fluid-tight seal of the proximal end of the syringe barrel. In addition, the syringe may comprise a closure device attached to the distal end of the syringe barrel, the closure device having an outlet engaging portion sealingly engaging and closing a distal open outlet end of the syringe system to prevent leakage of the liquid (aqueous) formulation such as a botulinum toxin formulation.

The “closure device” within the meaning of the present invention broadly refers to any means for closing and sealing the open outlet end of a syringe to prevent leakage. Within the present invention, the term “open outlet end” generally refers to any distal open end of a syringe that is in fluid communication with the barrel lumen. The “closure device” may, for example, be a “cap” or “tip cap” that is removed and replaced by a needle prior to use, or a sealing means like a needle shield in case of a syringe with a removable or permanent needle. To use the prefilled syringe, the tip cap, needle shield or other type of closure device are removed, optionally a needle is attached (if not already present), and the plunger tip or piston is advanced in the barrel to inject the contents of the barrel.

The closure device (e.g., tip cap or needle shield) may be a unitary member and is usually made from a flexible and/or resilient polymeric material (e.g., an elastomer), at least a portion of which contacts and seals the distal opening of the syringe (referred to as the “outlet engaging portion”). Alternatively, the closure device may have an outer cap made of a rigid plastic material that is coupled to a flexible and/or resilient inner cap made of a flexible and resilient polymeric material (e.g., an elastomer), wherein at least a portion of the inner cap contacts and seals the distal opening of the syringe (referred to as the “outlet engaging portion”).

If the syringe comprises said plunger stopper and said capping device and is filled with a formulation to be dispensed, e.g., an aqueous formulation such as an aqueous protein formulation or, more specifically, an aqueous botulinum toxin formulation, the syringe is a “pre-filled syringe”. As used herein, the term “prefilled syringe” refers to a syringe which is filled with a composition (e.g., a drug composition) prior to distribution to the end user. A prefilled syringe commonly includes a drug containment container forming part of a syringe body (i.e. a syringe barrel), a plunger stopper (and usually a plunger rod) to seal one open end of the syringe and for expelling the drug, and a closure device (e.g., a tip cap or a needle shield) on the outlet end of the syringe (e.g., the open end of the syringe tip or of a pre-mounted needle or cannula) to seal the distal outlet opening.

Within the context of the present invention, the outlet engaging portion of the closure device of the botulinum toxin prefilled syringe system, e.g. of a tip cap or a needle shield, is generally of an elastomeric material (e.g., wherein the elastomeric material is preferably a styrene-butadiene copolymer, a butyl rubber or a butyl rubber-isoprene rubber blend, and said butyl rubber is preferably a halogenated butyl rubber) optionally having a coating (e.g., a fluoropolymer coating, silicone coating, crosslinked silicone coating etc.) on an outer surface thereof such that the liquid (aqueous) formulation like an aqueous botulinum toxin formulation contacts only said coating during storage and/or injection.

The plunger stopper is preferably made of an elastomeric material (e.g., a butyl rubber such as a halogenated butyl rubber, in particular a chloro or bromo butyl rubber) and optionally has a coating on at least a portion of the plunger stopper such that the liquid (aqueous) formulation, e.g., an aqueous botulinum toxin formulation, contacts only said coating during storage and/or injection. Suitable coatings include polypropylene, polyethylene, parylene (e.g., parylene N, parylene C and parylene HT), silicone, crosslinked silicone and, preferably, fluoropolymer coatings (e.g., fluorinated ethylene-propylene such copolymer as tetrafluoroethylene-hexafluoropropylene copolymer (FEP)), fluorinated ethylene-ethylene copolymers (e.g., ethylene tetrafluoroethylene copolymer (ETFE) like FluroTec®).

In accordance with the present invention, the syringe may further comprise a plunger rod. The plunger rod may be fixed to the plunger stopper by any suitable means or may be integrally formed. Preferably, the plunger rod has a first mating member which engages a second mating member of the plunger stopper to removably connect the plunger rod to the plunger stopper. The rod is not particularly limited and may be designed to withstand sterilization. It is, however, also possible that the rod, which is mounted on the stopper from the outside, is not sterilized. Typically, the rod is made of a plastic material such as an ethylene vinyl acetate (EVA) copolymer or a polypropylene.

The syringe may further comprise an aqueous formulation, in particular an aqueous protein formulation such as an aqueous botulinum toxin formulation. In this case, the syringe is a pre-filled, ready-to-use syringe. The term “aqueous formulation”, as used herein has the meaning as defined herein above. Furthermore, the term “aqueous protein formulation” or “aqueous botulinum toxin formulation”, as used herein, is not particularly limited and may refer to an aqueous suspension, aqueous dispersion, aqueous emulsion and is preferably an aqueous solution. The aqueous protein formulation may comprise one or more additional substances like salts, buffers, surfactants, stabilizing proteins, amino acids, polyols, sugars (monosaccharides, disaccharides, oligosaccharides, and polysaccharides) and the like. The protein of the aqueous protein formulation is preferably a proteinaceous neurotoxin, more preferably a botulinum toxin. The pH of the aqueous protein formulation is typically in the range of 5.5 to 8.0 or 6.0 to 8.0, preferably 6.5 to 7.5, more preferably 6.8 to 7.4.

Preferred aqueous botulinum toxin formulations for use herein comprise water, botulinum toxin (e.g., the neurotoxic component of botulinum toxin free of complexing proteins, preferably of type A, or a botulinum toxin complex, preferably of type A) at a concentration of, for example, 1 to 1000 U/ml or 10 to 300 U/ml or 10 to 150 U/ml, a salt (e.g., sodium chloride) in a concentration such as 0.5% to 1.5% w/v, a stabilizing protein (e.g., albumin such as BSA or HSA) at a concentration such as 0.001% to 4% w/v, 0.01% to 3% w/v, 0.1% to 1% w/v or, particularly, 0.01% to 0.5% or 0.05 to 0.25%, and optionally a sugar (e.g., a mono- or disaccharide, such as glucose, fructose, galactose, trehalose, sucrose and maltose) at a concentration such as 0.1% to 2% w/v, or optionally a buffer (e.g., histidine, aspartate, glycine, glutamate, proline, taurine, methionine, serine, tyrosine, tryptophan or phosphate, particularly a histidine or phosphate buffer, more particularly a histidine buffer) at a concentration such as 1 to 100 mmol.

A further preferred aqueous botulinum toxin formulation for use herein essentially consists of water, botulinum toxin (e.g., the neurotoxic component of botulinum toxin type A free of complexing proteins), sodium chloride, sucrose, and albumin (e.g., human serum albumin; HSA). The concentration of the mentioned components may be in the following ranges: 10 to 200 U/ml or 30 to 125 U/ml (botulinum toxin), 0.5% to 1.5% w/v or 0.7% to 1.1% w/v (sodium chloride), 0.1% to 2% w/v or 0.2% to 1% w/v (sucrose), 0.01% to 1% w/v, 0.05% to 0.5% w/v, 0.1% to 3% w/v or 0.5% to 1.5% w/v (HSA). A further preferred botulinum toxin formulation for use herein is a Xeomin® solution, e.g., reconstituted with physiological saline (0.9% sodium chloride), including, e.g., 20 to 150 U/ml of the neurotoxic component of botulinum toxin type A. Another preferred botulinum toxin formulation for use herein is a Botox® or Dysport® solution, e.g., reconstituted with physiological saline (0.9% sodium chloride).

An alternative preferred aqueous botulinum toxin formulation comprises botulinum toxin (e.g. botulinum neurotoxin of serotype A), a surfactant (e.g., a non-ionic surfactant such as polysorbate), an amino acid selected from tryptophan (e.g. L-tryptophan) and tyrosine (e.g. L-tyrosine), a buffer comprising sodium, chloride phosphate ions, and optionally potassium ions, wherein said aqueous formulation has a pH between 5.5 and 8, preferably between 6.0 and 7.5, and is preferably free of animal derived proteins such as albumin, in particular free of bovine serum (BSA) or human serum albumin (HSA). Preferably, said aqueous formulation comprises 4 to 10000, preferably 10 to 2000, LD50 units of botulinum neurotoxin per mL, 0.001 to 15% v/v polysorbate (preferably 0.05 to 0.2% v/v polysorbate 80), 0.1 to 5 mg/ml tryptophan (preferably 0.1 to 5 mg/mL tryptophan), 10 to 500 mM NaCl (preferably 25 to 300 mM NaCl), 1 to 50 mM KCl (preferably 1 to 10 mM KCl), 1 to 100 mM sodium phosphate (preferably 2 to 50 mM sodium phosphate), and has a pH of between 5.5 and 8.

A still further alternative preferred aqueous botulinum toxin formulation comprises botulinum toxin (in non-complex form or in a complex form with a protein, e.g., in a concentration of 50 to 5,000 U/mL), polysorbate (e.g. polysorbate 20), and at least a proteinaceous amino acid (e.g. methionine and/or isoleucine). For example, the aqueous botulinum toxin formulation may comprise botulinum toxin, 0.01 to 50 mg polysorbate 20 per 100 units of botulinum toxin, and 0.5 to 100 μmol methionine per 100 units of botulinum toxin. For example, the aqueous botulinum toxin formulation may comprise methionine in concentration from 0.5 to 100 mM, preferably 25 to 75 mM, and polysorbate 20 in a concentration from 0.01 to 50 mg/mL, preferably from 0.1 to 2.5 mg/mL. Said aqueous botulinum toxin formulation may have a pH in the range of from 5.5 to 8.0 or 6.0 to 7.5.

In a fifth aspect, the present invention relates to a kit comprising a pre-filled syringe that comprises a syringe barrel of the present invention, and optionally instructions for use of said pre-filled syringe system.

Preferably, the pre-filled syringe is filled with an aqueous formulation, in particular an aqueous protein formulation such as an aqueous botulinum toxin formulation.

In a sixth aspect, the present invention relates to the use of a syringe of the present invention in therapy or aesthetic medicine, i.e., in a therapeutic method or in cosmetic applications.

For example, if the syringe is a botulinum toxin pre-filled syringe, the syringe can be used for treating a disease or condition caused by or associated with hyperactive cholinergic innervation of muscles or exocrine glands in a patient including, but not limited to, dystonia, spasticity, paratonia, diskinesia, focal spasm, strabismus, tremor, tics, migraine, sialorrhea and hyperhidrosis. Furthermore, the botulinum toxin pre-filled syringe may be used in the cosmetic treatment of wrinkles of the skin and facial asymmetries.

The present invention is further illustrated by the following Examples.

### EXAMPLES

The examples below illustrate the manufacturing of a syringe according to the present invention having a targeted thickness of the silicone layer for enhanced syringe functionality.

### Example 1

Manufacturing of syringes with varying silicone layer thicknesses

A 1 ml long standard glass syringe from Gerresheimer which is spray-siliconized (for details see Example 2; in the following referred to as “spray-siliconized standard syringe”) was subjected to mechanical and chemical treatments for reducing the thickness of the silicone layer. Specifically, the silicone layer was reduced by the following two methods:
1. In the first method, the silicone layer was reduced by mechanical treatment. A Novapure plunger stopper, connected to a rod, was inserted into the barrel, moved to the tip of the empty syringe and removed again from the barrel. This procedure was repeated multiple times, e.g., 10×, 50×, or 100×. With each cycle, a thin layer of the siliconization was gradually withdrawn from the inner surface of the barrel. The more removal cycles, the thinner the resulting remaining silicone layer of the syringe.
2. In the second method, an empty syringe was filled with an organic solvent, i.e. methylene chloride (or dichloromethane, DCM). The organic solvent was in direct contact with the silicone layer, resulting in the silicone layer to become dissolved at the contact surface. After a predetermined exposition time, the methylene chloride was removed from the syringe barrel, and the emptied syringe rested a couple of hours to allow the residual solvent to evaporate.

The silicone layer thickness and distribution before and after the mechanical and chemical treatments were measured using a device known as Bouncer (Unchained Labs, CA, USA). Bouncer automatically captured 45 data points along the 45 mm length of the syringe barrel. The syringe was automatically rotated in 60° increments to capture 6 lines. In total, 270 measurements were obtained from the syringe.

The results of the mechanical silicone removal are summarized in Table 1 below. The results show that there is a significant reduction of layer thickness after 25 cycles or more and that the resulting layer thickness decreases with a higher number of removal cycles. The best reduction results were seen after 100 cycles. After 100 cycles, the silicone layer thickness distribution is such that the lowest thicknesses are at about 40 nm and the thickest layer thicknesses at about 80 nm. Thus, the average silicone layer thickness is <80 nm.

TABLE 1
Mechanical reduction of silicone layer thickness

| Initial layer | Layer thickness after removal of plunger stopper for | | | | | |
|---|---|---|---|---|---|---|
| thickness* | 1x | 5x | 10x | 25x | 50x | 100x |
| 300 | >200 | >200 | >200 | <80 | <80 | <80 |
| nm | nm | nm | nm | nm | nm | nm |

*of the spray-siliconized standard syringe (see Table 3)

With respect to the organic solvent treatment, it can be seen from Table 2 below that there is a significant layer removal after a very short exposition time of only 30 seconds, with an extensive layer removal after 60 seconds. Longer expositions times of minutes or even in the hour range do not lead to any significant improvement to a thinner layer.

TABLE 2
Reduction of silicone layer thickness
by exposition to methylene chloride

| | |
|---|---|
| Initial layer | Layer thickness after exposition for |

| thickness* | 30 s | 60 s |
|---|---|---|
| 300 nm | <40 nm | <25 nm |

*of the spray-siliconized standard syringe (see Table 3)

As a comparison, the silicone layer thickness of a commercial baked-on siliconized glass syringe obtained from Gerresheimer (see Example 2 for details; in the following referred to as “baked-on siliconized standard syringe”) was measured in the same manner as described above. The results are shown in Table 3 together with the layer thicknesses of the spray-siliconized standard syringe, the 100×mechanically treated syringe, and the methylene chloride/60s treated syringe.

TABLE 3
Comparison of silicone layer thicknesses of standard syringes
and mechanical/chemical treated syringes of the invention

| Syringe | Mean layer thickness | |
|---|---|---|
| Baked-on siliconized standard syringe | <30-40 | nm |
| Spray-siliconized standard syringe | about 300 | nm |
| 100x mechanical treated syringe | <80 | nm |
| Methylene chloride/60 s treated syringe | <25 | nm |

It was found that the baked-on siliconized standard syringe has a much lower siliconization layer thickness of <30-40 nm at a minimal distance from the flange at which the inner surface of the barrel comes into contact with the syringe contents, if filled (e.g., at about 12 mm distance from the flange) and distances longer than said minimal distance, as compared to the spray-siliconized standard syringe (about 300 nm). Further, the layer thickness of the 100×mechanically treated syringe fall in between the high layer thickness of the spray-siliconized standard syringe and the low layer thickness of the baked-on siliconized standard syringe, and the methylene chloride/60s treated syringe has a mean silicone layer thickness of <25 nm.

The above results show that the present invention enables the manufacturing of syringes having a targeted silicone layer thickness which is neither too low nor too high. Lowering the silicone film thickness relative to the spray-siliconized standard syringe is expected to provide an elevated stability of the syringe content, in particular of an aqueous formulation such as an aqueous protein formulation, because the probability of formation of silicone droplets migrating into the formulation and exerting a destabilizing effect on agents such as protein agents is decreased. On the other hand, the thickness of the siliconization layer is still thick enough to achieve good gliding forces and thus a smooth travel of the plunger stopper through the syringe barrel, as shown in Example 2.

### Example 2

### Gliding Forces of Different Siliconized Syringes

For gliding force measurements, a Gerresheimer 1 ml long glass syringe (i.e. the “spray-siliconized standard syringe” of Example 1, i.e. Gerresheimer 1 ml long glass syringe, spray-siliconized, Luer Lock with TELC, inserted West 7025/65, grey) was combined with a West Novapure plunger stopper (RTU Plunger Stopper Novapure for 1.0 ml syringe, West 4023/50 grey, Flurotec coated, siliconized, Westar washed and sterilized)

The syringe barrels were treated using the methods described in Example 1, i.e.
by mechanical modification with 100 removal cycles, and
by organic solvent exposition with a contact time of 60 s and compared to the untreated spray-siliconized standard syringe and the untreated baked-on siliconized standard syringe (Gerresheimer 1 ml long glass syringe, baked-on siliconized, Luer lock with TELC, inserted Datwyler FM27/0, grey), each combined with the above-mentioned Novapure stopper (i.e. the same standard syringes as used in Example 1) at t=0 (TO, i.e. before storage) and after storage for 6 and 16 weeks under accelerated aging conditions of 40° C. and 75% relative humidity (RH). During the accelerated aging test, the syringes were stored horizontally.

The gliding force as a parameter of the performance of the syringe system was measured using a Mecmesin Multitest 2.5-xt force testing system (Mecmesin GmbH, Germany) combined with a Mecmesin ILC 100N load cell and Emperor force software. A 32Gx½″ test needle (Steriject PRE-32013, TSK Laboratory, Japan) was fitted to the syringe tip for the force measurements. Testing was carried out at room temperature (about 20° C.) and at a displacement speed of 100 mm/min into air. All measurements were performed using filled syringes, i.e. no “empty syringe measurements” were carried out. For the measurements, the syringes were filled with an aqueous botulinum toxin solution comprising 0.9 wt. % NaCl, 1 mg/ml human serum albumin and 4.7 mg/ml sucrose.

The force required for gliding the plunger stopper and plunger were recorded and presented as force versus displacement plots. The “mean gliding force” was determined from gliding force measurements of up to 15 identically prepared and stored pre-filled syringes to take into account the variability of single measurements. The results are shown in Table 4.

The gliding force (GF) was determined from the force vs. displacement plots:
gliding force (GF): the gliding force corresponds to the highest force between 1 mm and 35 mm plunger displacement distance (distance from flange) in Newton (N). The GF is automatically determined by the measurement device, but is checked manually since, for example, it might exceptionally be that with a high break loose force at 1 mm there is still a falling force slope, which could falsify the result for the gliding force.

TABLE 4
Gliding forces of siliconized syringes
after storage at 40° C./75% RH

| | | Mean gliding force | |
|---|---|---|---|
| | | after storage (40° | |
| | Mean gliding | C./75% RH) for | |
| Sample syringe | force at T0 | 6 weeks | 16 weeks |
| Spray-silicon. standard syringes | 4.9N | 5.8N | 6.0N |
| Baked-on silicon. standard syringes | 7.2N | 9.2N | 10.0N |
| Mechanically modified syringes | 6.2N | 6.8N | 6.4N |
| Methylene chloride treated syringes | 6.5N | 6.8N | 9.3N |

As can be seen from Table 4, the mechanically treated and methylene chloride treated syringes have gliding forces lying between the relatively high gliding force of baked-on siliconized syringes and the relatively low gliding force of spray-siliconized syringes. It it noted that the stopper used in the measurements is known to have good gliding characteristics and, thus, it is expected that the stability changes become even more pronounced if another typical commercial stopper, coated or uncoated, is used instead.

The above results show that the present invention enables the production of alternative siliconization layer thicknesses that are between the layer thicknesses of spray-siliconized and baked-on siliconized syringes. The silicone layer thicknesses provided according to the present invention are not too low to result in inadequate gliding forces and at the same time are not too high to result in compatibility (destabilizing) issues with the syringe content, in particular with an aqueous protein formulation.

1. A syringe barrel having a proximal end, a distal end and a sidewall extending therebetween, the sidewall having a cylindrical interior surface defining at least a portion of a chamber for receiving an aqueous formulation, wherein the cylindrical interior surface is coated with a silicone layer having a thickness of about 5 nm to about 100 nm.

2. The syringe barrel of claim 1, wherein the silicone layer has a thickness of about 40 nm to about 100 nm, about 40 nm to about 80 nm, about 45 nm to about 80 nm, or about 50 nm to about 70 nm, or wherein the silicone layer has a thickness of about 5 nm to about 70 nm, about 5 nm to about 50 nm, about 5 nm to about 30 nm, about 10 nm to about 35 nm, or about 15 nm to about 30 nm.

3. The syringe barrel of claim 1 or 2, wherein the silicone layer is a spray-on silicone layer that has been treated to reduce its thickness.

4. The syringe barrel of any one of claims 1 to 3, wherein the syringe barrel is made of glass, or wherein the distal end of the syringe barrel includes a tip having a passage extending therethrough and/or the proximal end of the syringe barrel includes an outwardly extending flange.

5. A method for manufacturing a syringe barrel according to any one of claims 1 to 4, the method comprising:
(a) providing a syringe barrel having a proximal end, a distal end and a sidewall extending therebetween, the sidewall having a cylindrical interior surface defining at least a portion of a chamber for receiving an aqueous formulation, wherein the cylindrical interior surface is coated with a silicone layer having an initial thickness,
(b) reducing the initial thickness of the silicone layer to between about 5 nm to about 100 nm or between about 30 nm and about 100 nm by exposing the cylindrical interior surface of the syringe barrel to an organic solvent or by subjecting the cylindrical interior surface of the syringe barrel to mechanical treatment, and
(c) recovering the syringe barrel.

6. The method of claim 5, wherein the silicone layer of the syringe barrel provided in step (a) is spray-on siliconized and/or has a thickness of about 120 to about 450 nm or about 150 nm to about 350 nm.

7. The method of claim 5 or 6, wherein the organic solvent is a hydrocarbon solvent, a halogenated hydrocarbon solvent, an ether solvent or a polar aprotic solvent, preferably a hydrocarbon solvent or a halogenated hydrocarbon solvent, more preferably a chlorinated hydrocarbon solvent including dichloromethane (DCM).

8. The method of any one of claims 5 to 7, wherein the cylindrical interior surface of the syringe barrel is exposed to the organic solvent for 5 seconds to 300 seconds.

9. The method of claim 5 or 6, wherein the mechanical treatment used in step (b) comprises the use of a thickness-reducing component configured to be slidably positioned inside the syringe barrel and providing a friction contact with the silicone layer coated on the cylindrical interior surface of the syringe barrel, and wherein the thickness-reducing component is used for performing multiple removal cycles, the multiple removal cycles comprising moving the thickness-reducing component to the distal end of the syringe barrel and then moving the thickness-reducing component to the proximal end of the syringe barrel and optionally removing the thickness-reducing component from the syringe barrel, repeating the moving steps and optional removing step until a silicone layer of the desired thickness within the range of 30 nm to 100 nm has been formed.

10. The method of claim 9, wherein in step (b) a plunger stopper is used as the thickness-reducing component and the number of removal cycles is preferably 10 to 1000, more preferably 20 to 500, 30 to 200 or 40 to 100.

11. A syringe barrel prepared by the method according to any one of claims 5 to 10.

12. A syringe comprising the syringe barrel according to any one of claims 1 to 4 and 11, optionally further comprising
a plunger stopper slidably positioned inside the syringe barrel and providing a fluid-tight seal of the proximal end of the syringe barrel;
a closure device attached to the distal end of the syringe barrel, the closure device having an outlet engaging portion sealingly engaging and closing a distal open outlet end of the syringe to prevent leakage, and optionally
an aqueous formulation, particularly an aqueous protein formulation and more particularly an aqueous botulinum toxin formulation.

13. A kit comprising a pre-filled syringe that comprises a syringe barrel according to any one of claims 1 to 4 and 11, and optionally instructions for use of said pre-filled syringe, the pre-filled syringe being preferably filled with an aqueous formulation, particularly an aqueous protein formulation and more particularly an aqueous botulinum toxin formulation.

14. A syringe of claim 12 for use in a therapeutic method.

15. Use of a syringe of claim 12 in cosmetic applications.
